(19) United States
(12) Patent Application Publication
(10) Pub. No.: US 2026/0212970 A1
(43) Pub. Date: Jul. 23, 2026

(54) DIAGNOSTIC DATA USER INTERFACES

(71) Applicant: Franklin-AI Pty Ltd, Sydney, New South Wales (AU)

(72) Inventors: Vanessa CHEUNG, Sydney, New South Wales (AU); Stuart WISHART, Sydney, New South Wales (AU); Lan HUANG, Sydney, New South Wales (AU); Ming Yiu KWONG, Sydney, New South Wales (AU); Peter DASSOS, Sydney, New South Wales (AU)

(21) Appl. No.: 19/142,107

(22) PCT Filed: Dec. 22, 2023
(86) PCT No.: PCT/AU2023/051358

§ 371 Date: Jun. 20, 2025

(30) Foreign Application Priority Data
Dec. 22, 2022 (AU) 2022903981

(51) Int. Cl. G16H 10/40; G16H 15/00; G16H 40/67

(52) U.S. Cl. CPC G16H 10/40; G16H 15/00; G16H 40/67

(57) ABSTRACT

Methods and graphical user interfaces for receiving, analysing and reporting medical diagnostic data and findings are disclosed. A screen display of the graphical user interface associates a plurality of slide indicators and diagnostic indicators. A user may navigate to a second screen display, the second screen display also including diagnostic indicators and providing user input mechanisms to specify a change or addition to the diagnostic findings.

### RELATED APPLICATION

This application is related to Australian patent application number 2022903981 filed on 22 Dec. 2022, the contents of which are incorporated herein by reference in their entirety in jurisdictions that allow such incorporations.

### TECHNICAL FIELD

Aspects of the present disclosure are directed to computer systems, methods, and graphical user interfaces and more specifically to computer systems, methods, and graphical user interfaces for receiving, analysing, and reporting medical diagnostic data and findings.

### BACKGROUND

When a medical test (e.g., MRI scan, physical examination) identifies a mass in a patient's body, a referring doctor may ask the patient to undergo a biopsy to acquire tissues for a formal pathological diagnosis. The result may lead to the diagnosis of a variety of conditions such as infection, inflammation, and tumours and tumour-like processes that can be benign or malignant. Similarly, bodily tissue collected as part of a medical procedure may also be analysed for pathological diagnosis.

When patient body tissue is collected, one or more samples of potentially diseased tissues are removed for pathology examination. The tissue sample collected from a common (non-differentiable) physical location is known as a “specimen”. In the process leading to microscopic examination, a specimen is processed, embedded in a wax block, and sliced into very thin layers and each layer is placed on a glass slide. The glass slides are then examined by a pathologist under a microscope, either optical or digital. A pathologist reports their findings to the referring doctor in the form of a “pathology report” to assist them in confirming the diagnosis of the diseased tissue and guiding therapy. Examination of specimens enables a pathologist to determine if a tissue is noncancerous or cancerous. Cancerous tumours are malignant, which means they can grow and spread to other parts of the body. Noncancerous or benign tumours can still grow but will not spread.

Generating pathology reports for cancer can be tedious and time-consuming work. A typical pathology examination involves reviewing several specimens by analysing the pathology slides associated with each specimen, finding and categorising cancerous and other patterns of concern, collating slide-level and specimen-level findings to make overall diagnostic findings for a case, and reporting the findings in a pathology report.

A pathologist may receive pathology slides at a computer system. The computer system receives details of the pathology slides and other information identifying and concerning the subject and presents the pathology slides to the pathologist. The pathologist may also record their findings in the computer system, for communication and further action as required for the subject.

### SUMMARY

According to a first aspect of the present disclosure a computer-implemented method is provided including receiving or generating pathology diagnostic findings data in relation to a plurality of pathology slides; causing the display of a first screen display, the first screen display including a plurality of slide indicators identifying a plurality of pathology slides and one or more first diagnostic indicators of diagnostic findings associated with one or more of the plurality of pathology slides, wherein the slide indicators and diagnostic indicators are based on the received or generated diagnostics findings data.

In some embodiments, while causing the display of the first screen display, the method further includes receiving first user input specifying a pathology slide from the plurality of pathology slides; in response to the first user input causing the display of a second screen display, the second screen display including one or more second diagnostic indicators of diagnostic findings associated with the specified pathology slide; while causing the display of the second screen display, receiving second user input specifying a change or addition to the diagnostic findings; and in response to the second user input, updating the second screen display and the received or generated diagnostics finding data to reflect the change or addition.

In some embodiments the second diagnostic indicators comprise at least the one or more first diagnostic indicators.

In some embodiments the computer-implemented method further includes: receiving a third user input after receiving the second user input and in response to the third user input generating and/or displaying a report based on the updated received or generated diagnostics finding data.

In some embodiments the computer-implemented method further includes: receiving image data representing an image of each of the pathology slides, including a first image of the pathology slide that is associated with the one or more second diagnostic indicators of diagnostic findings associated with the specified pathology slide; and causing the display of the first image.

In some embodiments the pathology diagnostic findings data includes data indicating one or more parts of the first image associated with the one or more second diagnostic indicators and wherein the computer-implemented method includes causing the display of one or more indicators of the one or more parts of the first image.

In some embodiments the one or more indicators of the one or more parts of the first image include one or more colour or pattern codes applied to the image at the one or more parts of the first image.

In some embodiments the one or more indicators of the one or more parts of the first image include one or more boxes applied to the image around the one or more parts of the first image.

In some embodiments the computer-implemented method further including, while displaying the one or more indicators of the one or more parts of the first image, receiving a further user input and in response to the further user input, displaying the first image without the one or more indicators of the one or more parts of the first image.

In some embodiments the computer-implemented method includes causing the display of the first image while causing the display of the second screen display.

According to another aspect of the present disclosure a computer implemented method is provided including receiving or generating pathology diagnostic findings data in relation to a plurality of pathology slides with an associated plurality of specimens, wherein the plurality of pathology slides include a first pathology slide and a second pathology slide associated with a first specimen of the plurality of specimens and a third pathology slide not associated with the first specimen; displaying a first screen display including first indicators of the pathology findings data for the first pathology slide and/or the second pathology slide and while displaying the first screen display receiving first user input that specifies the first specimen; in response to the first user input displaying a second screen display including second indicators of the pathology findings data for the first specimen, the second indicators representing an aggregation of the pathology findings data for the first pathology slide and the second pathology slide; while causing the display of the first display screen, receiving second user input specifying a change or addition to the diagnostic findings; and in response to the second user input, updating the first screen display and the second screen display and the received or generated diagnostics finding data to reflect the change or addition.

In some embodiments the computer-implemented method further includes: while causing the display of the second display screen, receiving third user input specifying a change or addition to the diagnostic findings; and in response to the third user input, updating the second screen display and the received or generated diagnostics finding data to reflect the change or addition.

In some embodiments the computer-implemented method further includes: receiving a further user input and in response to the further user input generating and/or displaying a report based on the updated received or generated diagnostics finding data.

According to another aspect of the present disclosure a computer implemented method is provided including receiving image data of images of a plurality of pathology slides, including a first image of a first of the pathology slides; receiving pathology diagnostic findings data in relation to the plurality of pathology slides, including the first pathology slide, the pathology findings data indicating one or more parts of the first pathology slide associated with a pathology diagnostic finding defined by the pathology diagnostic findings data; receiving first user input that specifies the first pathology slide; in response to the first user input displaying a mini-viewer screen including the first image and graphical representation of the one or more parts of the first pathology slide associated with the pathology diagnostic finding.

In some embodiments the computer-implemented method further includes receiving second user input and in response to the second user input displaying the first image without the graphical representation of the one or more parts of the first pathology slide associated with the pathology diagnostic finding.

In some embodiments the computer-implemented method further includes receiving third user input and in response to the third user input displaying the first image with the graphical representation of the one or more parts of the first pathology slide associated with the pathology diagnostic finding.

In some embodiments the graphical representation includes highlighted regions, corresponding to the one or more parts of the first pathology slide associated with the pathology diagnostic finding, overlayed on the first image.

In some embodiments the computer-implemented method further includes: receiving further user input indicating acceptance, rejection, or addition of a diagnostic finding; in response to the further user input modifying the pathology diagnostic findings data to reflect the change indicated by the computer user.

In some embodiments the computer-implemented method further includes: receiving further user input specifying a said graphical representation and a magnify request; in response to the further user input magnifying the region on the first image associated with the specified graphical representation.

In some embodiments the computer-implemented method further includes: displaying a summarised report of the diagnostic findings including a summarised report for each specimen, most important diagnostic findings across all specimens, and visual representation of areas affected by cancerous tissues.

In some embodiments the visual representation of areas affected by cancerous tissues includes a plurality of biopsy sample locations and diagnostic finding indicators associated with each biopsy sample location.

In some embodiments the summarised report for each specimen includes a pattern bar chart where the pattern bar chart represents the overall area in the specimen affected by cancerous tissues and contains distinct areas for each type of cancerous pattern detected in the specimen.

In some embodiments a computer system configured to perform the methods described above.

In some embodiments non-transient computer readable memory including instructions to cause a computer processor to perform the methods described above.

Further aspects of the present disclosure and further embodiments of the aspects described above will become apparent from the following description, made with reference to the accompanying drawings.

### BRIEF DESCRIPTION OF THE DRAWINGS

FIG. 1 depicts a computer processing system environment.

FIG. 2A depicts a sample pathology review application screen with AI findings activation overlay window.

FIG. 2B depicts AI findings activation overlay window.

FIG. 3 depicts an intra-case triage overlay screen.

FIG. 4A depicts a slide diagnostic review overlay screen.

FIG. 4B depicts another view of a slide diagnostic review overlay screen.

FIG. 5 depicts a specimen-level report overlay window.

FIG. 6A depicts a slide mini-viewer window in slide preview mode.

FIG. 6B depicts a slide mini-viewer window in region list review mode.

FIG. 6C depicts a slide mini-viewer window in region preview mode.

FIG. 7 depicts a diagnostic report overlay window.

FIG. 8 depicts a graphical diagnostic report overlay window.

FIG. 9 to FIG. 11 depict flowcharts representing methods that may be run or executed by a computer processing system, for example the system of FIG. 1.

Where different figures include the same or similar features, the same reference numerals are used to indicate those features. The figures showing windows and screens are shown in greyscale. This is not intended to be limiting and the figures may be rendered in colour, for example as shown in the corresponding figures of Australian patent application number 2022903981.

While the description is amenable to various modifications and alternative forms, specific embodiments are shown by way of example in the drawings and are described in detail. It should be understood, however, that the drawings and detailed description are not intended to limit the invention to the particular form disclosed. The intention is to cover all modifications, equivalents, and alternatives falling within the scope of the present invention as defined by the appended claims.

### DETAILED DESCRIPTION

In the following description numerous specific details are set forth in order to provide a thorough understanding of the present invention. It will be apparent, however, that the present invention may be practiced without these specific details. In some instances, well-known structures and devices are shown in block diagram form in order to avoid unnecessary obscuring.

When commencing evaluation of a set of pathology slides of a patient, a pathologist is generally required to assume equal importance for all pathology slides, because each slide may reveal tissue patterns consequential in diagnosing the type of cancer a patient may have and the treatment that must follow. Therefore, a pathologist may sequentially review all slides. However, it is more likely that a subset of all slides are of increased importance or significance for diagnostic purposes relative to others, and some slides may contain no regions of interest. Prioritising the review of slides of potentially more importance may therefore assist a pathologist, for example by saving time in reaching a conclusion and/or facilitating an improvement in the accuracy of findings by allowing the pathologist to spend more time examining more consequential slides.

In this disclosure reference is made to an example examination of pathology slides for evaluating the presence of prostate cancer. This is not intended to be limiting-the computer systems, methods and graphical user interfaces may be used to facilitate and assist examinations in other cancer or tissue types. Additionally, the current disclosure does not intend to introduce a new standard for pathology reporting. Further, the utility of the disclosed computer systems, methods and graphical user interfaces is not confined to a particular standard for pathology reporting.

When a biopsy is carried out in the process of diagnosing for prostate cancer, the collected specimens are pathologically examined in order to determine the prognosis (i.e., predicting the likelihood or expected development of the cancer). Together with other parameters, a Gleason grading system may be used to determine the stage of the disease, predict prognosis, and help guide therapy. Microscopic appearance of collected specimens is the basis for a pathologist to determine a Gleason pattern (i.e., a number between 1 and 5) for the cancer where a higher number generally indicates a greater risk and higher mortality. The Gleason patterns identified across the specimens are then aggregated, following standard clinical process, to calculate the Gleason score and Gleason Grade Group for the case. Gleason scoring and grading system is widely accepted and used for prostate cancer clinical decision making.

A pathologist may assign several Gleason patterns to each slide associated with a specific region in the specimen depending on the cancerous patterns observed in the corresponding tissues. After examining all the slides of a specimen, the pathologist may identify up to the three most dominant cancer patterns across all the slides of the specimen. The final Gleason score for a specimen is calculated by following a standard clinical diagnostic heuristic which considers the two Gleason patterns with the largest areas and whether any Gleason pattern 5 has been identified.

When a pathologist reviews the slides of a specimen, they may find other issues of concern. By way of example, other types of malignancies (e.g., intraductal carcinoma, metastasised cancer from another region of the body), prognostic factors (e.g., perineural invasion), and other conditions (e.g., prostatic intraepithelial neoplasia, basal cell hyperplasia, prostatic atrophy, partial atrophy, or prostatic inflammation) can be found. It is also possible that a specimen contains an atypical small acinar proliferation (ASAP) that is suspicious for but not diagnostic of malignancy.

As noted earlier, in the process of examining specimens, a pathologist may create and analyse a plurality of slides for one or more specimens. A pathological examination may demand that a pathologist analyses between 1 to over 100 slides to identify or confirm a diagnosis and produce a pathology report, which can be a laborious and time consuming undertaking. One reason for the slowness of the examination process is that a pathologist may sequentially examine all slides, as they have no prior indication as to whether each slide will contain any noteworthy abnormalities leading to a diagnosis. For example, if it takes an average of 3 minutes to analyse each slide and there are a total of 20 slides for all the specimens, it will take a pathologist approximately an hour to examine all the slides (i.e., find patterns of interest, associate Gleason grades to all specimens, calculate Gleason scores, and note other findings worthy of reporting). One aspect of case reporting which can cause an increase in diagnostic time, and pathologist fatigue, is when the first slides inspected don't have clear cancer patterns and the pathologists spends a large amount of time and mental energy thoroughly reviewing the slides to ensure that a pattern is not missed only to find that the last slide in the case contains an easily identified Gleason pattern. All such findings must then be reported in a coherent pathology report, the preparation of which will require additional time.

The present disclosure is concerned with computer systems, methods and graphical user interfaces that may assist a pathologist to more efficiently or effectively examine pathology slides and specimens and prepare pathology reports. The computer system may generate or receive and utilise output from artificial intelligence (AI) technology applied to analyse a collection of pathology slides associated with one or more specimens. In particular the AI technology is configured to find potential patterns of interest, and/or assign potential Gleason patterns, based on a computer-readable representation of each slide. The AI technology may output an estimate of a Gleason score for each specimen, and/or output possible textual interpretations for findings, that can be used in producing a pathology report. The AI technology may form part of the computer system, or may be implemented by another computer system, with the output received and processed in line with the present disclosure. The present disclosure is made primarily with reference to this application. However, the present disclosure also has other applications, for example to receiving preliminary findings from a trainee pathologist or an assistant to a pathologist and presenting those for review to a pathologist to make any required changes or additions.

In some embodiments the computer system provides a graphical user interface including a summary of findings for all specimens and slides (i.e., an intra-case triage). The user interface is navigable from the summary to data representing specific slides, enabling a pathologist to attend to the most important slides and specimens as a matter of priority. In some embodiments the computer system, by indicia on the graphical user interface, brings to the attention of a pathologist the potential areas of interest on each slide where potentially cancerous patterns can be found. In some embodiments, the computer system displays in the graphical user interface a potential Gleason score to be assigned to each specimen. In some embodiments the computer system provides a graphical user interface with a pre-populated report or part thereof, for example including data and textual interpretations of findings in a manner consistent with known pathology report templates. The graphical user interface provides interactive elements that allow a pathologist to review and modify information presented to them and generate a pathology report they approve of.

In the following paragraphs, screen displays or parts thereof are described. The screen displays are part of a graphical user interface of a software application (referred to herein as a “slide review application”), run or executed by a computer processing system (e.g., computer processing system 100 below), for use in a review of pathology slides and specimens.

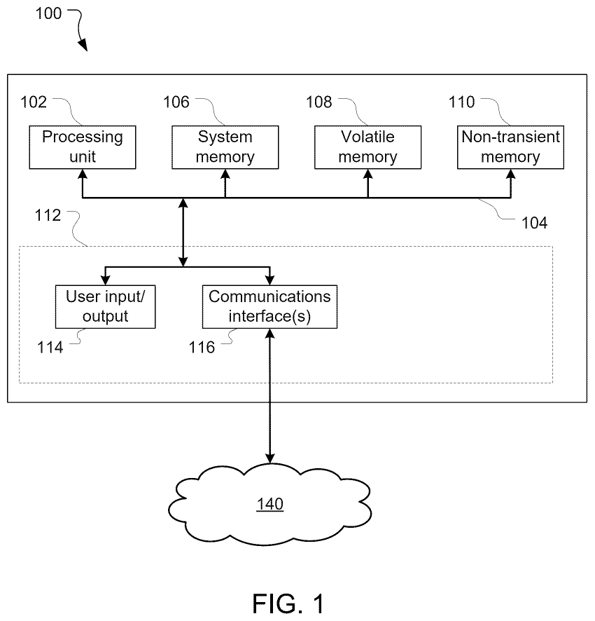

FIG. 1 shows a block diagram of a computer processing system 100 configurable to implement embodiments and/or features described herein. System 100 is a general purpose computer processing system. It will be appreciated that FIG. 1 does not illustrate all functional or physical components of a computer processing system. For example, no power supply or power supply interface has been depicted, however system 100 will either carry a power supply or be configured for connection to a power supply (or both). It will also be appreciated that the particular type of computer processing system will determine the appropriate hardware and architecture, and alternative computer processing systems suitable for implementing features of the present disclosure may have additional, alternative, or fewer components than those depicted.

Computer processing system 100 includes at least one processing unit 102—for example a general or central processing unit, a graphics processing unit, or an alternative computational device. Computer processing system 100 may include a plurality of computer processing units. In some instances, where a computer processing system 100 is described as performing an operation or function all processing required to perform that operation or function will be performed by processing unit 102.

Through a communications bus 104, processing unit 102 is in data communication with a one or more computer readable storage devices which store instructions and/or data for controlling operation of the processing system 100. In this example system 100 includes a system memory 106 (e.g., a BIOS), volatile memory 108 (e.g., random access memory such as one or more DRAM applications), and non-volatile (or non-transitory) memory 110 (e.g., one or more hard disks, solid state drives, or other non-transitory computer readable media). Such memory devices may also be referred to as computer readable storage media (or a computer readable medium).

System 100 also includes one or more interfaces, indicated generally by 112, via which system 100 interfaces with various devices and/or networks. Generally speaking, other devices may be integral with system 100, or may be separate. Where a device is separate from system 100, connection between the device and system 100 may be via wired or wireless hardware and communication protocols, and may be a direct or an indirect (e.g., networked) connection.

Wired connection with other devices/networks may be by any appropriate standard or proprietary hardware and connectivity protocols, for example Universal Serial Bus (USB), eSATA, Thunderbolt, Ethernet, HDMI, and/or any other wired connection hardware/connectivity protocol.

Wireless connection with other devices/networks may similarly be by any appropriate standard or proprietary hardware and communications protocols, for example infrared, sBluetooth, Wi-Fi; near field communications (NFC); Global System for Mobile Communications (GSM), Enhanced Data GSM Environment (EDGE), long term evolution (LTE), code division multiple access (CDMA-and/or variants thereof), and/or any other wireless hardware/connectivity protocol.

Generally speaking, and depending on the particular system in question, devices to which system 100 connects-whether by wired or wireless means-include one or more input/output devices (indicated generally by input/output device interface 114). Input devices are used to input data into system 100 for processing by the processing unit 102. Output devices allow data to be output by system 100. Example input/output devices are described below, however, it will be appreciated that not all computer processing systems will include all mentioned devices, and that additional and alternative devices to those mentioned may well be used.

For example, system 100 may include or connect to one or more input devices by which information/data is input into (received by) system 100. Such input devices may include keyboards, mice, trackpads (and/or other touch/contact sensing devices, including touch screen displays), microphones, accelerometers, proximity sensors, GPS devices, touch sensors, and/or other input devices. System 100 may also include or connect to one or more output devices controlled by system 100 to output information. Such output devices may include devices such as displays (e.g., cathode ray tube displays, liquid crystal displays, light emitting diode displays, plasma displays, touch screen displays), speakers, vibration applications, light emitting diodes/other lights, and other output devices. The screen displays described herein may be provide by a display of the system 100, or by a display of another suitable computer system. System 100 may also include or connect to devices which may act as both input and output devices, for example memory devices/computer readable media (e.g., hard drives, solid state drives, disk drives, compact flash cards, SD cards, and other memory/computer readable media devices) which system 100 can read data from and/or write data to, and touch screen displays which can both display (output) data and receive touch signals (input).

System 100 also includes one or more communications interfaces 116 for communication with a network, such as network 140 of environment 100. Via a communications interface 116 system 100 can communicate data to and receive data from networked devices, which may themselves be other computer processing systems.

System 100 may be any suitable computer processing system, for example, a server computer system, a desktop computer, a laptop computer, a netbook computer, a tablet computing device, a mobile/smart phone, a personal digital assistant, or an alternative computer processing system.

System 100 stores or has access to computer applications (also referred to as software or programs)—i.e., computer readable instructions and data which, when executed by the processing unit 102, configure system 100 to receive, process, and output data. Instructions and data can be stored on non-transitory computer readable media accessible to system 100. For example, instructions and data may be stored on non-transitory memory 110. Instructions and data may be transmitted to/received by system 100 via a data signal in a transmission channel enabled (for example) by a wired or wireless network connection over interface such as 112.

Applications accessible to system 300 will typically include an operating system application such as Microsoft Windows™, Apple macOS™, Apple iOS™, Android™, Unix™, or Linux™ System 100 also stores or has access to applications which, when executed by the processing unit 102, configure system 100 to perform various computer-implemented processing operations described herein.

In some cases part or all of a given computer-implemented method will be performed by a single computer processing system 100, while in other cases processing may be performed by multiple computer processing systems in data communication with each other.

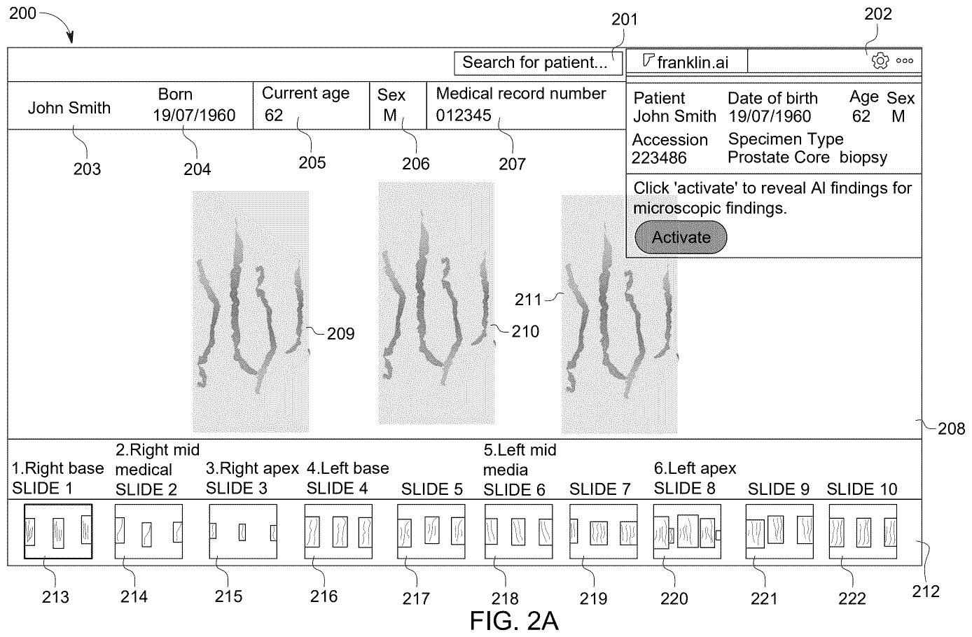

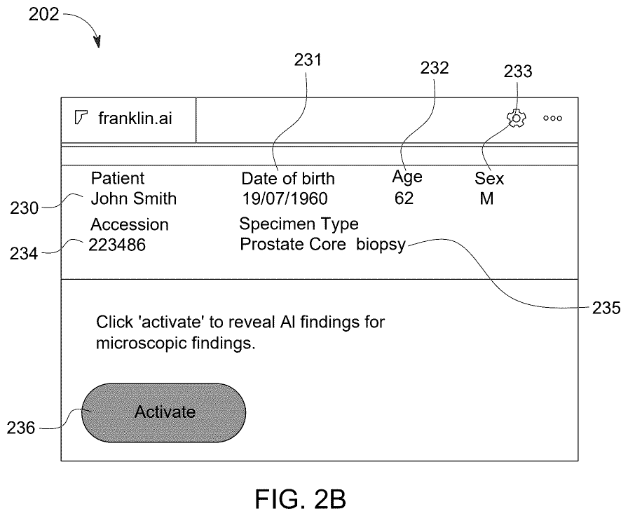

FIG. 2A shows a screen display 200 of a slide review application used by a pathologist to review pathology slides, which consists of a range of user interface elements. In this embodiment, the slide review application causes the screen display 200 to contain elements including a patient search bar 201, patient identification information (e.g., name 203, date of birth 204, current age 205, sex 206, and medical record number 207), a slide image view panel 208, specimen levels 209-211, slide thumbnail preview bar 212, and slide thumbnails 213-222. Data defining the patient identification information and the specimen slide scans was received by the computer processing system running the slide review application.

The screen display 200 also includes an AI findings activation overlay window 202, which enables a pathologist to request the output of the computer system running the AI technology described earlier to be selectively presented. The AI findings activation overlay window 202 may be a native part of the slide review application, or may be generated by another software application. For example, the AI findings activation overly window 202 may be a GUI of a software application that also implements the AI technology.

FIG. 2B shows a larger preview of the AI findings activation overlay window 202. The AI findings activation overlay window 202 contains patient identification information (e.g., name 230, date of birth 231, current age 232, sex 233, and case accession number 234). A pathologist can visually match patient identification information 203-207 with the information in the AI findings activation overlay window 202 to confirm that AI findings are correctly matched with this pathology examination. Accession number 234 is an identification number for the collection of specimens under examination and in this embodiment is the same as medical record number 207 of the patient. Specimen type 235 indicates the type or purpose of specimen collection, which in this instance is prostate core biopsy. By clicking on activation button 236, a pathologist instructs the computer system to show a summary of findings for all specimens and slides (i.e., an intra-case triage). For example, selecting the activation button may cause the slide review application to request the output from the AI technology, which output may have been previously generated or which may be generated “on demand”, in response to receipt of data indicating that the activation button 236 has been selected.

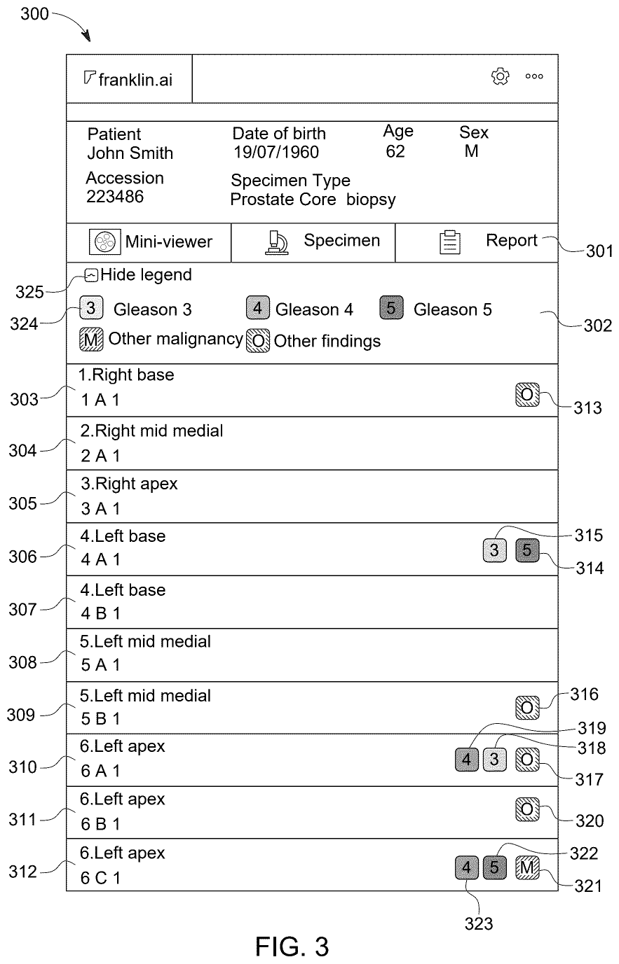

FIG. 3 shows an intra-case triage overlay screen 300. The intra-case triage overlay screen 300 replaces the AI findings activation overlay window 202 following and responsive to selection of the activation button 236. In other embodiments the intra-case triage overlay screen 300 is displayed responsive to other events and/or is displayed by default (i.e., without further user input) when the slide image view panel 208 is displayed. The intra-case triage overlay screen 300 may be displayed simultaneously with the slide image view panel 208.

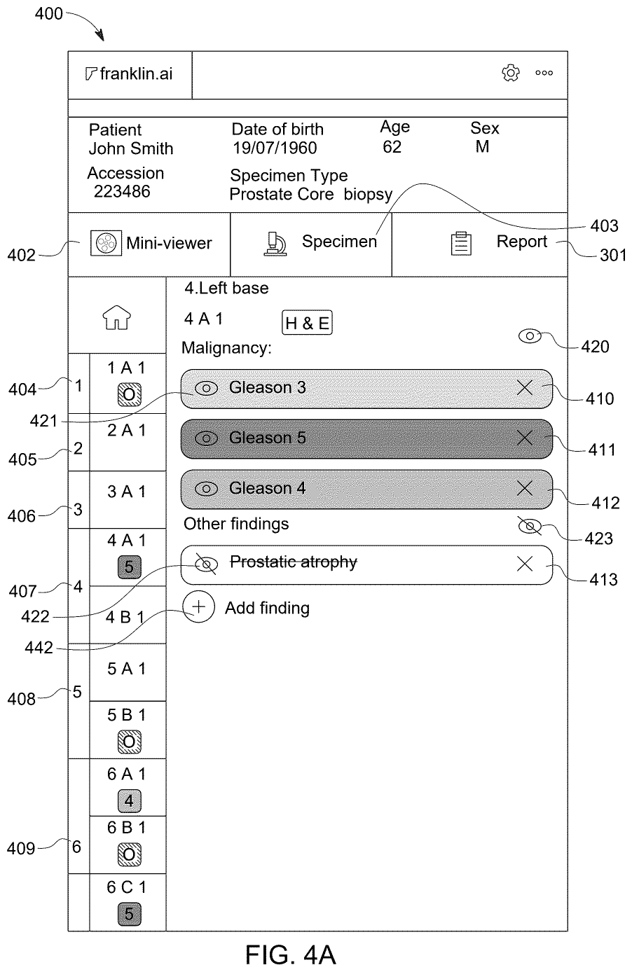

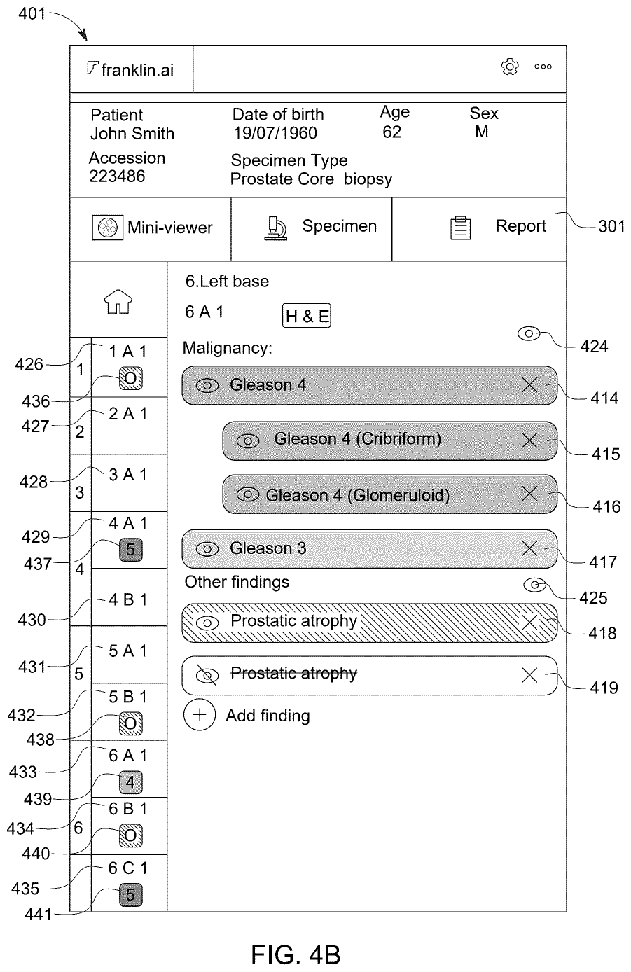

The intra-case triage overlay screen 300 provides a pathologist with information to conduct a review of a plurality of, up to all of, the slides for one or more specimens for a patient, together with diagnostic findings by the AI technology associated with each slide. In the embodiment shown the intra-case triage overlay screen 300 includes report preview control 301, legend area 302, slide detail rows 303-312, and diagnostic finding indicators 313-323. Screen 300 may also include patient identification information as explained in relation to FIG. 2B but not marked again with reference numerals in FIG. 3. Screen 300 also includes mini-viewer preview control and specimen details control, which are shown in an inactive state. These controls are explained below in relation to FIGS. 4A and 4B where they are both shown in an active state (i.e., controls 402 and 403).

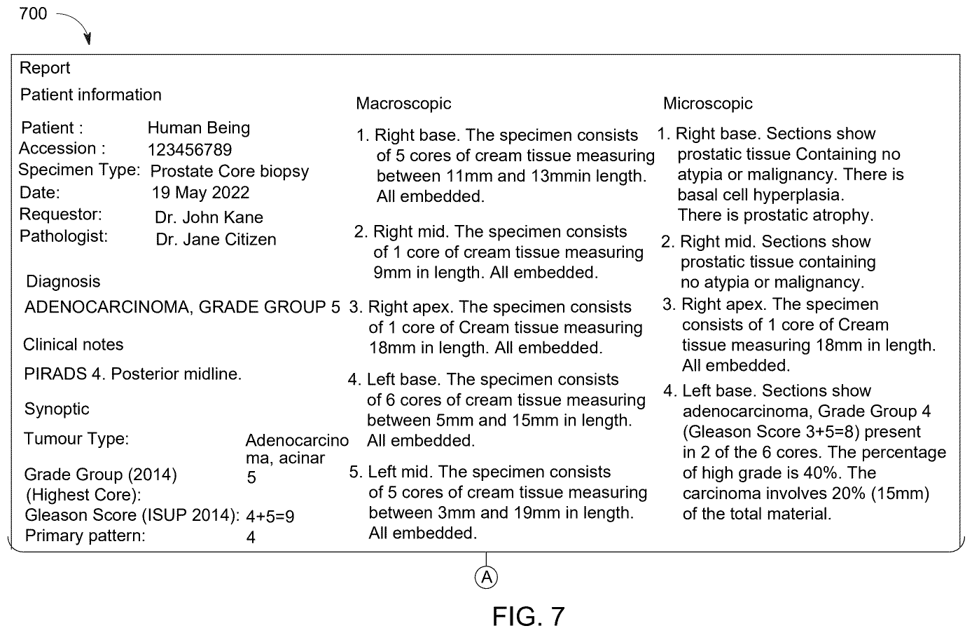

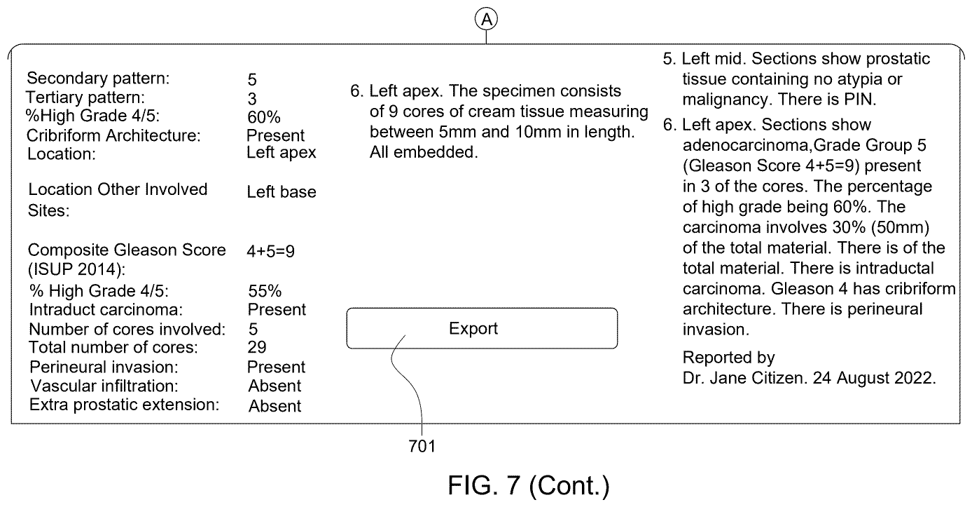

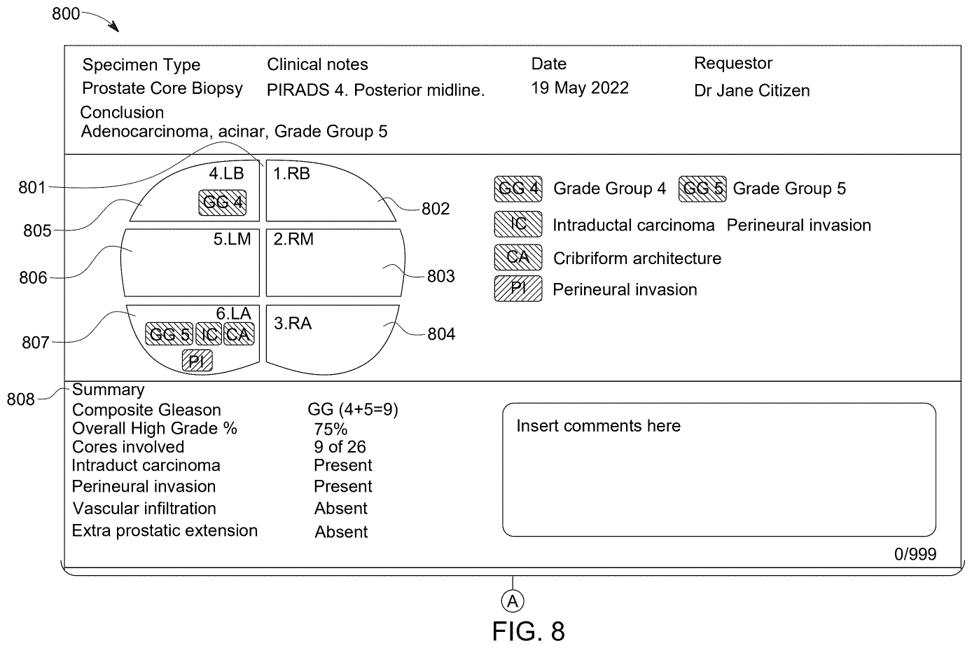

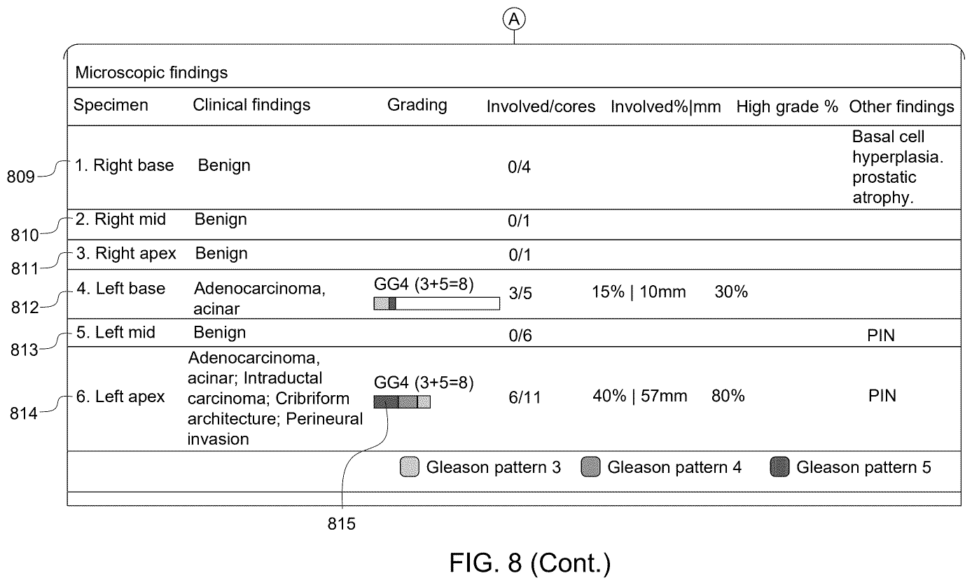

A pathologist can click on report preview control 301 to open or close a preview window showing the pathology report for the current examination. The details of such preview window are presented later in relation to FIGS. 7 and 8.

Legend area 302 contains guides for a set of diagnostic finding indicators 313-323. By way of example, the rounded-corner rectangle 324 containing number 3 indicates detection of Gleason pattern 3. In FIG. 3, slides detail rows 306 and 310 are labelled with rectangles similar to rectangle 324, which indicates that Gleason pattern 3 is detected in slides 4 A 1 and 6 A 1. Similarly, slides 6 A 1 and 6 C 1 are marked as containing Gleason pattern 4, slides 4 A 1 and 6 C 1 are marked as containing Gleason pattern 5, slide 6 C 1 is marked as containing other malignancies, and slides 1 A 1, 4 A 1, 5 B 1, 6 A 1 and 6 B 1 are marked as containing other findings. In some embodiments each Gleason pattern number is associated with and displayed with a distinct colour or pattern, in both the guides and in the diagnostic finding indicators.

In some embodiments the legend area 302 dynamically displays guides dependent on the AI technology output. In particular, guides are provided only for findings that are present in the AI technology output. In this example, a total of only 5 of more than 5 legend items available for inclusion in the legend area 302 are included in legend area 302 because only such 5 types of findings are associated with slide detail rows 303-312. In other embodiments legend area 302 may contain a different number of guiding signs (e.g., a comprehensive list of all possible signs regardless of the actual findings in the slides), be displayed only after a user's request (e.g., by clicking on a show/hide control element such as element 325), or not be present at all. In yet other embodiments such guiding information that is presented in legend area 302 may be displayed to a user in response to the user's request (e.g., by pointing a mouse pointer on any one of diagnostic finding indicators 313-323).

As noted earlier, intra-case triage screen 300 enables a pathologist to review, in some embodiments in one page, a plurality of and up to all slides related to all specimens collected in relation to an examination. The diagnostic information displayed by diagnostic finding indicators 313-323 assist a pathologist in determining a view on the relative importance of each slide and prioritising the order in which they should be examined.

By way of example, by reviewing diagnostic finding indicators 313-323 a pathologist is able to infer that slides 2 A 1, 3 A 1, 4 B 1, 5 A 1 are less likely to contain any diagnostic patterns and therefore have a lower priority for examination because the computer system has not provided any preliminary diagnostic finding information for these slides. On the other hand, the existence of a relatively high number of indicators 317-319 and 321-323 may assist a pathologist to associate a higher relative importance to slides 6 A 1 and 6 C 1 and therefore a higher priority for examination. The presence of a plurality of indicators 314-315 may also indicate that a higher priority should be given to slide 4 A 1. In addition to or instead of focussing on a number of indictors, the pathologist may take into account the Gleason Score communicated by the indicator, in this embodiment by both a number and a pattern or colour. So therefore, a pathologist may give slide 4 A 1 a higher priority than slide 6 A 1, even though slide 6 A 1 is displayed associated with more indicators. The user interface therefore provides tools for a pathologist to infer the relative importance of each slide for examination, which may lead to a more efficient use of the pathologist's time, for example by avoiding a sequential examination and allowing spending of more time on slides that are more likely to contain substantive findings.

The user interface may transition from the display of the intra-case triage screen 300 to the display of one or more slide diagnostic review screens responsive to a user input indicating one or more of slides. For example, a user may use a point and click device to select one of the slides within the intra-case triage screen 300. The user interface may then replace the intra-case triage screen 300 with a slide diagnostic review screen for the selected slide. Alternatively, the slide diagnostic review screen may be displayed simultaneously with the intra-case triage screen, for example beside the intra-case triage screen. In either case, the slide diagnostic review screen may be displayed simultaneously with the slide image view panel 208, for example as an overlay to the slide image view panel 208 (see e.g., FIG. 2A). The slide image view panel 208 may be updated, if needed, to display the selected slide or the selected slides presented in the slide diagnostic review screen or screens.

FIGS. 4A and 4B show two example slide diagnostic review overlay screens 400 and 401 respectively for slides 4 A 1 and 6 A 1. Each screen 400 and 401 may contain mini-viewer preview control 402, specimen details control 403, report preview control 301, specimen number indicators 404-409, diagnostic information bars 410-419, diagnosis visibility controls 420-425, slide number indicators 426-435, diagnostic indicators 436-441, and add findings control 442. In other embodiments one or more of these features is omitted and/or additional features added. Screens 400 and 401 may also include patient identification information as explained in relation to FIG. 2B but not marked again on FIG. 4.

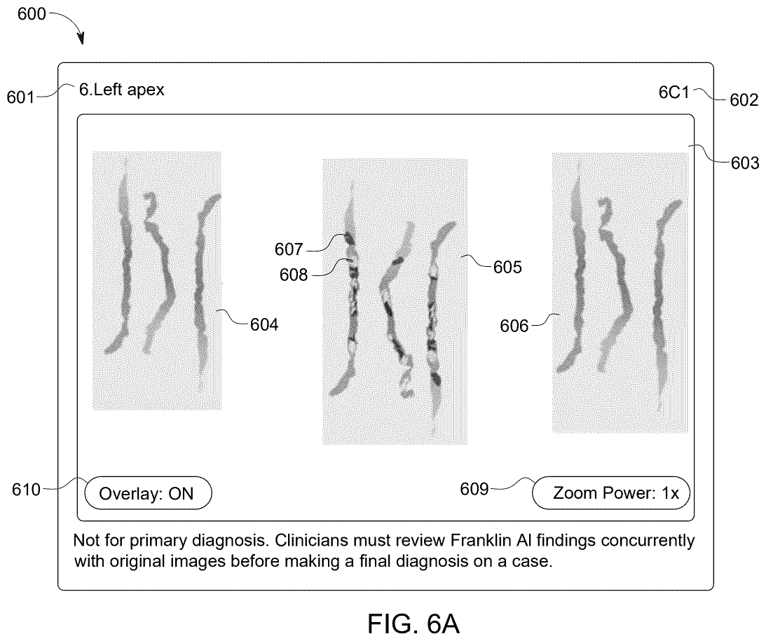

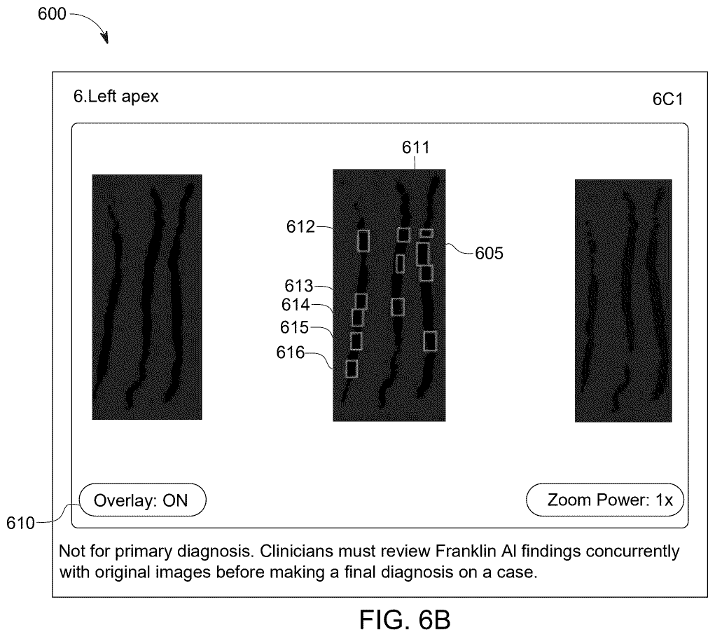

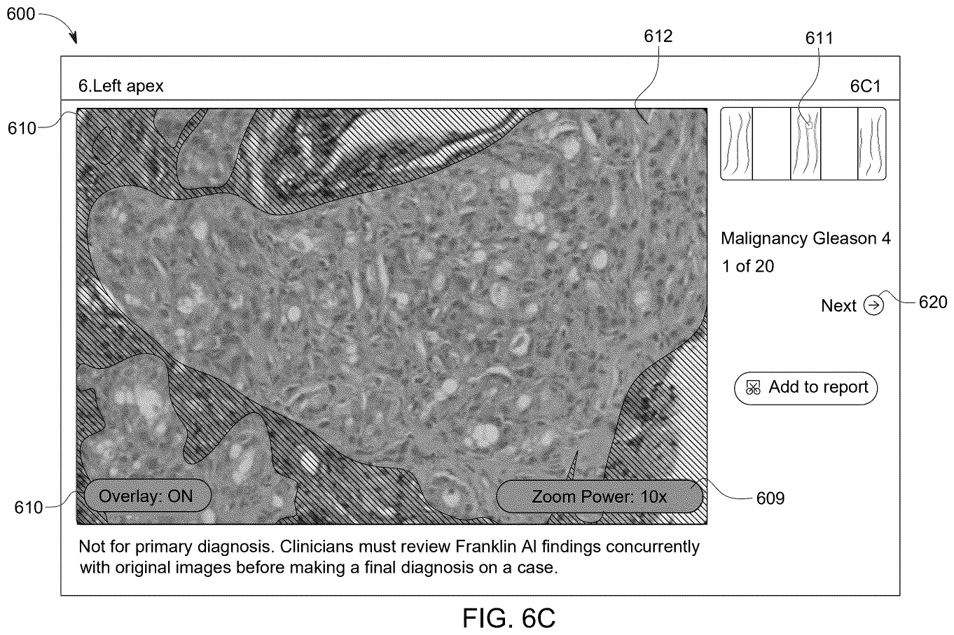

A pathologist can click on mini-viewer preview control 402 to open or close a preview window showing an image of tissue areas that the computer system has identified as being affected by various diagnostic findings. The image may be, for example, a magnified image or an image that is magnified responsive to a user input. The magnified image may be magnified relative to the image of the slide image view panel 208. The image may also or instead be an image modified to indicate sections of the image that are associated with the diagnostic indicators displayed on the slide diagnostic review screen. For example, sections of the image may be indicated in colours or patterns that match the colours or patterns indicating a Gleason score in the slide diagnostic review screen. The details of an example of such a preview window are presented later in relation to FIGS. 6A-6C below. In other embodiments the magnified image or the modified image to show sections associated with diagnostic indicators may be displayed in the slide image view panel 208, instead of or in addition to the mini-viewer preview control 402.

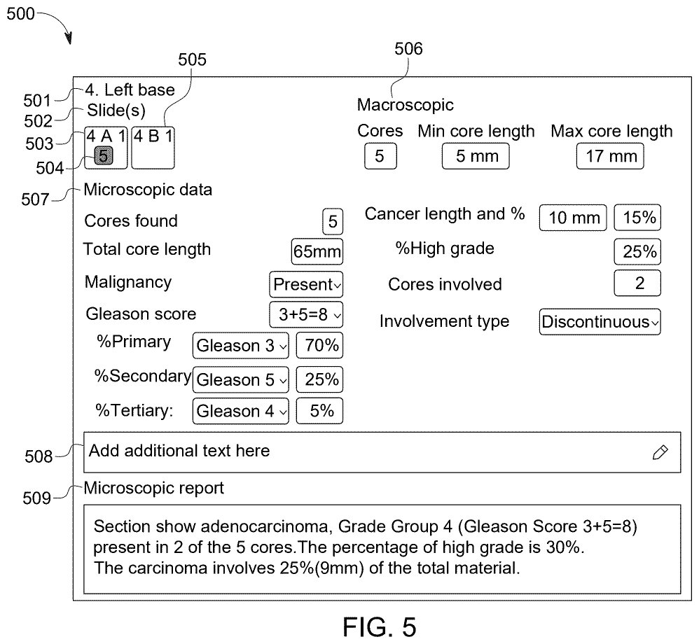

A pathologist can click on specimen details control 403 to open or close a window containing the overall findings related to a specimen. The details of such display window are presented later in relation to FIG. 5.

Report preview control 301 has a similar function to the same control on FIG. 3. Refer to the descriptions below in relations to FIGS. 7 and 8 for the details.

As noted earlier, there may be one or more specimens that a pathologist is required to review in a given pathology examination and each specimen may be associated with one or more slides. Specimen number indicators 404-409 show all specimens which are available to review. The corresponding slides are identified with slide number indicators 426-435.

In this embodiment, the association of specimens and slides is shown by the number of relevant specimen showing as a prefix to all its associated slide numbers. By way of example, specimen number 1 (indicator 404) is associated only with slide 1 A 1 (426), specimen number 4 (407) is associated with slides 4 A 1 (429) and 4 B 1 (430), and specimen number 6 (408) is associated with slides 6 A 1-6 C 1 (433-435). In other embodiments, the numbering system used for specimen and slide representation as well as the method of association between the two may vary. Several examples are possible such as not including specimen number indicators 404-409 in screens 400 and 401, representing the association between specimens and slides by other means (e.g., using a dedicated colour or pattern for each specimen and all its slides, or making specimen numbers as headings and the slide numbers as child items of each relevant heading).

In FIG. 4A, diagnostic information bars 410-413 are representatives of various types of diagnosis made by the computer system in relation to the slide currently under review (i.e., slide 4 A 1). Similarly, in FIG. 4B, diagnostic information bars 414-419 are representative of diagnosis in relation to slide 6 A 1. When the computer system is configured to detect patterns that identify a particular type of cancer (e.g., Cribriform or Glomeruloid) that are sub-categories of certain Gleason patterns, such information is also shown among the information bars (e.g., 415, 416). In other embodiments, such diagnostic information may be shown using other methods of visual presentations (e.g., by textual descriptors, or other visual clues supported with a guide as in FIG. 3).

When a pathologist examines a slide (e.g., slide 4 A 1 or 6 A 1), slide diagnostic review overlay screens (e.g., 400 and 401) show computer findings that enable the pathologist to direct their effort to slides identified as having significant diagnostic findings. The pathologist is able to reject a diagnostic finding, in which case the computer system removes the finding for the slide from its data records. The removal from the data records also causes a corresponding update to a generated pathology report (later explained in relation to FIGS. 7 and 8). In the example shown, the pathologist can reject a diagnostic finding by clicking on the ‘X’ sign on the corresponding diagnostic information bar 410-413. The diagnostic findings not rejected by the pathologist are deemed valid and will be presented in the pathology report. If the pathologist makes new findings which have not been identified by the computer system, they can add such findings using add findings control 442. The add findings control 442 causes the user interface to display a screen to enter the new finding and after the new finding is entered, it is included in the data records of the computer system for the slide, displayed in the slide diagnostic review screen for the slide, and included in the pathology report.

While reviewing a slide, each finding made by the computer system (e.g., Gleason pattern 3) is accompanied with an overlay highlight on the relevant sections of the tissues affected by that finding. This is further explained in relation to FIGS. 6A-6C below. Diagnostic visibility controls 420-425 enable the pathologist to cause the computer system to show/hide the overlay highlight or other indicator in relation to diagnostic finding by the computer system. By way of example, visibility control 420 and 424 enables showing/hiding of the slide overlays related to any type of malignancy, and visibility control 405 enables showing/hiding of slide overlay highlight related to Gleason score 3 patterns found in slide 4 A 1. Visibility control 422 and 423 show a change in the status of the control when the corresponding overlays are hidden.

Diagnostic finding indicators 436-441 show a determined most important diagnostic finding in relation to each slide. The determination may be made by the computer system, for example based on a predefined ordering of importance. When reviewing a slide, indicators 436-441 assist a pathologist in deciding the other slides to review and/or in what order. By way of example, a pathologist reviewing slide 4 A 1 as shown in the overlay window 400 will be able to see indicator 441 showing that slide 6 C 1 (435) contains Gleason pattern 5 and therefore may decide to review slide 6 C 1 as the next slide because of its relative importance. In this embodiment, diagnostic indicators 436-441 are shown using small rounded-corner rectangles containing a number or a letter. In other embodiments, such indicators could be shown using other graphical elements (e.g., text-only, coloured texts, patterned background).

FIG. 5 shows a specimen-level report overlay window 500 which presents diagnostic findings related to all slides of a specimen. Window 500 may include visual categories to group related reporting elements. In this embodiment, window 500 includes specimen label 501, slides section 502, macroscopic summary data section 506, microscopic data section 507, and microscopic report section 509.

In this example, window 500 represents diagnostic findings related to specimen 4 (i.e., left base). Slides section 502 includes a list of all slides associated with the currently presented specimen and diagnostic indicators corresponding to each slide that show the most prominent or important diagnostic related to each slide. In this example, specimen 4 is shown to be associated with two slides: slide 4 A 1 (503) with diagnostic indicator 504 and slide 4 B 1 (505) with no diagnostic indicators.

Macroscopic summary data section 506 contains accessioning data in relation to the currently presented specimen. In this embodiment, this data includes the number of cores covered by the specimen, and minimum and maximum core lengths. Typically, such data is provided by a pathology laboratory and maintained by the computer system for consistency and traceability.

Microscopic data section 507 includes standard pathology reporting elements such as, e.g., cores found in the specimen, total core length, whether malignancy is detected, Gleason score (including its primary, secondary, and tertiary grades), cancer length and percentage, percentage of high grade, cores involved, and involvement type.

After the computer system analyses all the slides related to each specimen, specimen-level findings of the computer system are used to prepopulate the user interface elements under microscopic data section 507. A pathologist is then able to review such findings and amend as they deem is required. Accordingly, the data section 507 contains user editable data fields. A pathologist is also able to add additional findings by using add finding control 508. In this example the add finding control 508 is a text field for the entry of any text the pathologist wishes to add. The text will be added to the pathology report. In some embodiments the text is added to microscopic report section 509 (see below) after being entered using add finding control 508.

The number of elements shown under microscopic data section 507 depend on the findings of the computer system. By way of example, in screen 500 it is shown that malignancy is detected in specimen 4 and therefore a corresponding Gleason score has been reported. Where no malignancy is detected in a specimen, screen 500 may be simplified by, e.g., prepopulating screen 500 such that malignancy control shows absence of malignancy and hiding all other controls related to reporting a detected malignancy (e.g., Gleason score, primary-tertiary grades etc).

Microscopic report section 509 contains a prepopulated description, generated by the computer system based on the prepopulated diagnostic findings in section 507. The description from the microscopic report section 509 is included in the pathology report generated by the computer system. When a pathologist makes amendments to the findings in section 507, the description in section 509 is automatically updated, thereby providing consistency across the whole report.

FIG. 6A shows an embodiment of a mini-viewer overlay window 600, which contains specimen indicator 601, slide number indicator 602, slide mini-preview panel 603, specimen level mini-previews 604-606, zoom power control 609, and overlay control 610.

Specimen indicator 601 and slide number indicator 602 correspondingly show the specimen number and slide number currently shown. Specimen level mini-preview 605 contains regions highlighted in various colours or patterns such as, e.g., highlighted region 607-608. Use of different colours or patterns to represent different Gleason patterns identified in a tissue enables a pathologist to quickly review the affected regions and decide which regions should be examined with a relatively higher priority. Receipt of selection by the pathologist of the overlay control 610 causes the computer system to turn on or off the overlay, allowing the pathologist to selectively review the original images without the overlay, in addition to the images with the overlay (as shown in FIG. 6A). Zoom power control 609 enables viewing the specimen level mini-previews in a greater level of detail.

FIG. 6B shows the mini-viewer overlay window 600 showing regions found to contain a specific Gleason pattern. In the example shown in FIG. 6B, a total of 12 regions are specified (e.g., 611-616) where a specific pattern has been detected (e.g., Gleason pattern 4).

FIG. 6C shows the mini-viewer overlay window 600 in a higher zoom level (e.g., 10× as shown by control 609). The mini-viewer overlay window shows the same highlighted regions as displayed in FIG. 6B (e.g., 611) where a specific coloured overlay (e.g., area 612 in FIG. 6C) shows the regions of interest. In FIG. 6C, the cross-hatched areas indicate areas without any overlay, which will be displayed in a different colour to the area 612. Different areas in FIG. 6C may also be differentiated by using various shades or patterns. In this embodiment, when such zoom level is specified by a pathologist using control 609, the regions affected by patterns are shown in the order of their importance. In this example, region 611 is deemed to be the most important diagnostic finding and is shown first in preview panel 603 as the first region. A pathologist may review each affected region by using control button 620. Alternatively or additionally, the computer system may be configured to allow the pathologist to select a part of the image, for example a highlighted section, and zoom into that section.

In this embodiment, window 600 has three modes of operation shown in FIGS. 6A-6C. In other embodiments, each such mode of operation could constitute a separate window. Less than all three modes may be implemented in other embodiments, or additional modes added.

FIG. 7 (shown across two pages indicated by connector “A”) shows diagnostic report overlay window 700, which contains a report generated by the computer system after a pathologist has completed the pathology examination, including the AI findings accepted by the pathologist. The report may follow a relevant prostate cancer reporting guidelines structure. The computer system also reflects the amendments made by a pathologist in previous steps (e.g., rejecting some findings and adding new ones), and populates window 700. Further modification of the report is possible in this window, allowing a pathologist to further refine the reported results. Export control 701 allows exporting the findings of the computer system, which may be in a standard pathology report format that may be used by other pathology software programs (e.g., pathology laboratory information systems).

FIG. 8 (shown across two pages indicated by connector “A”) shows graphical diagnostic report overlay window 800. This window is an alternative or additional form of showing a diagnostic report of FIG. 7 and like that report it includes findings of the AI accepted by the pathologist and any changes or additions to the findings. Various elements in window 800 intend to present diagnostic findings in a summarised and visual form that enable a faster review. Window 800 contains prostate biopsy template diagram 801, prostate biopsy location diagrams 802-807, case synoptic data section 808, specimen summary rows 809-814, and grade bar chart 815.

Prostate biopsy template diagram 801 contains a simplified visual representation of a prostate cross-section. Diagram 801 includes an approximate geometry of a prostate, which may be divided into separate parts. In this embodiment, diagram 801 contains prostate biopsy location diagrams 802-807 corresponding to right apex, right mid, right base, left apex, let mid, left base. In other embodiments, there may be a different sub-division of the prostate or other biopsy template diagrams corresponding to different tissue sample areas (e.g., liver, breast).

Prostate biopsy location diagrams 802-807 are presented such that the Gleason grade and other findings associated with each location can be easily understood through the diagnostic finding indicators associated with each location. In this embodiment, diagnostic finding indicators are shown as rectangles with red or blue filling. For example, prostate biopsy location diagram 807 (left apex) is associated with Gleason grade 5, intraductal carcinoma, cribriform architecture, and perineural invasion. In other embodiments, a combination of other colours, patterns and/or textual descriptions may be used as diagnostic finding indicators.

Case synoptic data section 808 contains case level data representing an overall assessment of the case based on the examination of all slides of all specimens. In this embodiment, this section includes data such as composite Gleason score and Gleason Grade Group, overall high grade percent, number of cores involved (i.e., affected by cancerous tumours), and whether certain types of diagnostic indicators are detected (e.g., intraduct carcinoma, perineural invasion, vascular infiltration, extra prostatic extension). In other embodiments, section 808 may contain a different number of data items.

Specimen summary rows 809-814 present a simplified summary of diagnostic findings across all sides of each specimen. By way of example, row 812 indicates that left base specimen's examination has found ⅜ of cores to be affected, Gleason Grade Group 4 is detected (primary pattern=3, secondary pattern=5, and Gleason score=8), and approximately 15% of specimen area is affected by cancerous patterns. In the example shown in window 800, the pathology report is produced by examining 6 specimens. In other examples, a different number of specimens may be examined in a pathology report and therefore reported in window 800.

Each of specimen summary rows 809-814 also includes a pattern bar chart (e.g., bar chart 815). Each bar chart visually represents the overall area in a specimen affected by cancerous tissues and contains distinct areas for each type of cancerous pattern detected. In this embodiment, different colour shades are used to represent distinct areas in a bar chart. For example, three shades of grey colour respectively represent Gleason patterns 3, 4 and 5. In other embodiments, colours and/or patterns may be used to represent visually distinct areas of bar charts. Size of each distinct area of a bar chart corresponds to the size of the tissue area affected by the corresponding cancerous pattern. By way of example, the bar chart located in row 812 indicates that approximately 15% of left base specimen is affected by two cancerous patterns wherein a relatively larger area is affected by Gleason pattern 3 and a relatively smaller area is affected by Gleason pattern 5.

Screen 800 may contain other user interface elements which are not shown in FIG. 8. By way of example, similar to screen 700, there may be an export control 701 in screen 800 with similar functionalities. Additionally, a pathologist may be provided with control elements in screen 800 to attach sample images (e.g., most important slides) to the pathology report.

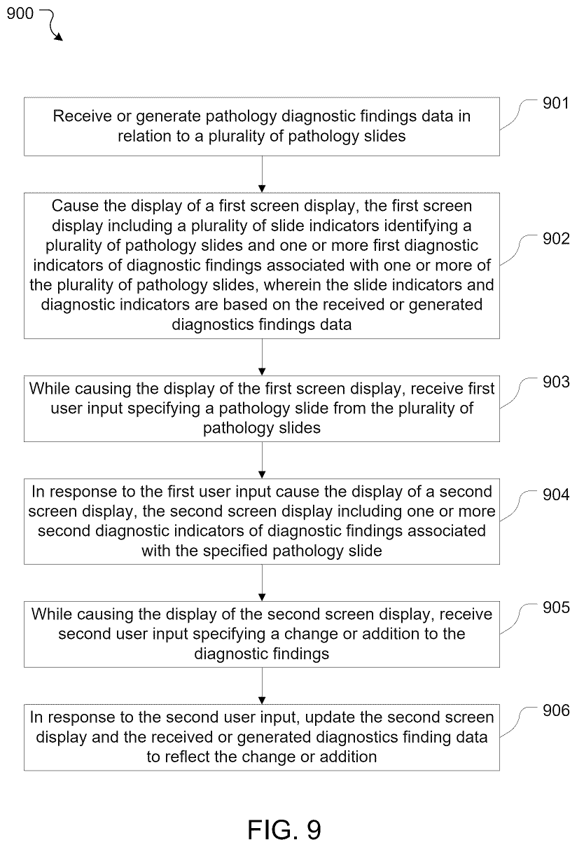

FIGS. 9-11 show flowcharts representing methods that may be run or executed by a computer processing system, such as computer processing system 100 described in reference to FIG. 1. Certain embodiments of the methods cause the display of the screen displays described herein. The computer processing system may run or execute any one or more of the methods, in any combination. The computer processing system may perform additional steps or processes to those described in order to provide further functionality described herein.

In some embodiments, instructions to cause a processing unit, for example the processing unit 102, may be stored on non-transient memory 110. The instructions may be loaded into the volatile memory 108 and executed by the processing unit 102. In other embodiments, instructions may be received by computer processing system 100 through communications interface 116 and executed by processing unit 102.

Where user interaction is required for the operation of methods shown in FIGS. 9-11, computer processing system 100 may receive user input and send information to output through input/output device interface 114.

In FIG. 9, steps of method 900 are described. At step 901, computer processing system 100 receives or generates pathology diagnostic findings data in relation to a plurality of pathology slides. At step 902, processing unit 102 causes the display of a first screen display. The first screen display includes a plurality of slide indicators identifying a plurality of pathology slides. The first screen display includes also includes one or more first diagnostic indicators of diagnostic findings associated with one or more of the plurality of pathology slides, wherein the slide indicators and diagnostic indicators are based on the received or generated diagnostics findings data.

At step 903, while processing unit 102 is causing the display of the first screen display, computer processing system 100 receives first user input, for example through input/output device interface 114 or through communications interface 116, specifying a pathology slide from the plurality of pathology slides.

At step 904, in response to the first user input, processing unit 102 causes the display of a second screen display. The second screen display includes one or more second diagnostic indicators of diagnostic findings associated with the specified pathology slide.

At step 905, while processing unit 102 is causing the display of the second screen display, computer processing system 100 receives second user input, for example through input/output device interface 114 or through communications interface 116, specifying a change or addition to the diagnostic findings.

At step 906, in response to the second user input, processing unit 102 updates the second screen display and the received or generated diagnostics finding data to reflect the change or addition.

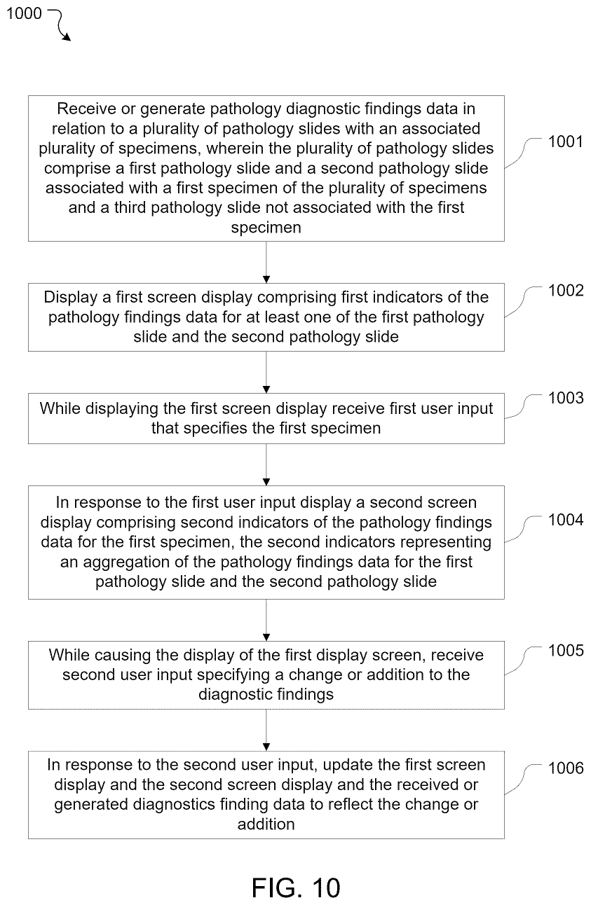

Turning to FIG. 10, steps of method 1000 are described. At step 1001, computer processing system 100 receives or generates pathology diagnostic findings data in relation to a plurality of pathology slides with an associated plurality of specimens. The plurality of pathology slides include a first pathology slide and a second pathology slide associated with a first specimen of the plurality of specimens and a third pathology slide not associated with the first specimen.

At step 1002, computer processing system 100 displays a first screen display including first indicators of the pathology findings data for the first pathology slide and/or the second pathology slide.

At step 1003, while displaying the first screen display, computer processing system 100 receives first user input, for example through input/output device interface 114 or through communications interface 116, that specifies the first specimen.

At step 1004, in response to the first user input, computer processing system 100 displays a second screen display including second indicators of the pathology findings data for the first specimen. The second indicators represent an aggregation of the pathology findings data for the first pathology slide and the second pathology slide.

At step 1005, while causing the display of the first display screen, computer processing system 100 receives second user input, for example through input/output device interface 114 or through communications interface 116, specifying a change or addition to the diagnostic findings.

At step 1006, in response to the second user input, processing unit 102 updates the first screen display and the second screen display and the received or generated diagnostics finding data to reflect the change or addition.

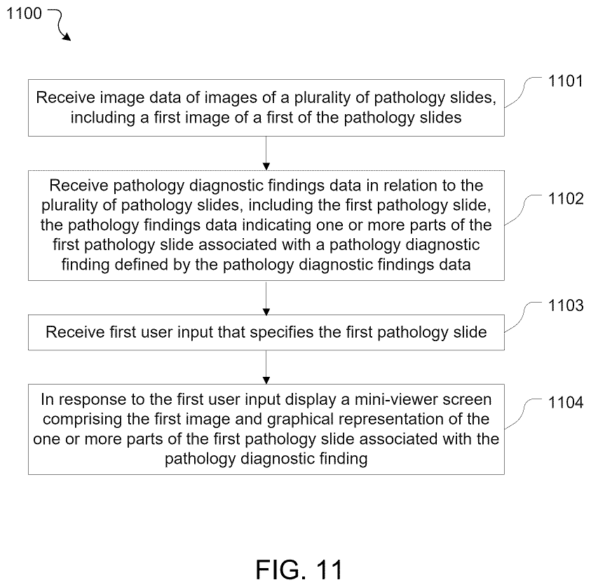

Turning to FIG. 11, steps of method 1100 are described. At step 1101, computer processing system 100 receives image data of images of a plurality of pathology slides, including a first image of a first of the pathology slides.

At step 1102, computer processing system 100 receives pathology diagnostic findings data in relation to the plurality of pathology slides, including the first pathology slide, the pathology findings data indicating one or more parts of the first pathology slide associated with a pathology diagnostic finding defined by the pathology diagnostic findings data. The reception of the image data and the pathology diagnostic findings data may be simultaneous, for example as a single communication of a set of data files, or sequentially as separate communications, in either order.

At step 1103, computer processing system 100 receives first user input, for example through input/output device interface 114 or through communications interface 116 that, specifies the first pathology slide.

At step 1104, in response to the first user input, computer processing system 100 displays a mini-viewer screen including the first image and graphical representation of the one or more part of the first pathology slide associated with the pathology diagnostic finding.

As used herein, including in the claims, except where the context requires otherwise, the term “comprise” and variations of the term, such as “comprising”, “comprises” and “comprised”, are not intended to exclude further additives, components, integers or steps. Additionally, the terms “first” and “second”, for example in “first screen display” and “second screen display” are intended to denote to individual instances of the feature referred to and are not intended to imply any temporal ordering or other temporal relationship. The term “and/or”, for example in “X and/or Y”, means X or Y or both.

It will be understood that the invention disclosed and defined in this specification extends to all alternative combinations of two or more of the individual features mentioned or evident from the text or drawings. All of these different combinations constitute various alternative aspects of the invention.

1. A computer-implemented method, comprising:
receiving or generating pathology diagnostic findings data in relation to a plurality of pathology slides;
causing the display of a first screen display, the first screen display including a plurality of slide indicators identifying a plurality of pathology slides and one or more first diagnostic indicators of diagnostic findings associated with one or more of the plurality of pathology slides, wherein the slide indicators and diagnostic indicators are based on the received or generated diagnostics findings data;
while causing the display of the first screen display, receiving first user input specifying a pathology slide from the plurality of pathology slides;
in response to the first user input causing the display of a second screen display, the second screen display including one or more second diagnostic indicators of diagnostic findings associated with the specified pathology slide;
while causing the display of the second screen display, receiving second user input specifying a change or addition to the diagnostic findings; and
in response to the second user input, updating the second screen display and the received or generated diagnostics finding data to reflect the change or addition.

2. The computer-implemented method of claim 1, wherein second diagnostic indicators comprise at least the one or more first diagnostic indicators.

3. The computer-implemented method of claim 1. further comprising:
receiving a third user input after receiving the second user input and in response to the third user input generating and/or displaying a report based on the updated received or generated diagnostics finding data.

4. The computer-implemented method of claim 3, further comprising:
receiving image data representing an image of each of the pathology slides, including a first image of the pathology slide that is associated with the one or more second diagnostic indicators of diagnostic findings associated with the specified pathology slide; and
causing the display of the first image.

5. The computer-implemented method of claim 4, wherein the pathology diagnostic findings data comprises data indicating one or more parts of the first image associated with the one or more second diagnostic indicators and wherein the method includes causing the display of one or more indicators of the one or more parts of the first image.

6. The computer-implemented method of claim 5, wherein the one or more indicators of the one or more parts of the first image comprise one or more colour or pattern codes applied to the image at the one or more parts of the first image.

7. The computer-implemented method of claim 5, wherein the one or more indicators of the one or more parts of the first image comprise one or more boxes applied to the image around the one or more parts of the first image.

8. The computer-implemented method of claim 5, further comprising, while displaying the one or more indicators of the one or more parts of the first image, receiving a further user input and in response to the further user input, displaying the first image without the one or more indicators of the one or more parts of the first image.

9. The computer-implemented method of claim 4, comprising causing the display of the first image while causing the display of the second screen display.

10. A computer-implemented method, comprising:
receiving or generating pathology diagnostic findings data in relation to a plurality of pathology slides with an associated plurality of specimens, wherein the plurality of pathology slides comprise a first pathology slide and a second pathology slide associated with a first specimen of the plurality of specimens and a third pathology slide not associated with the first specimen;
displaying a first screen display comprising first indicators of the pathology findings data for the first pathology slide and/or the second pathology slide and while displaying the first screen display receiving first user input that specifies the first specimen;
in response to the first user input displaying a second screen display comprising second indicators of the pathology findings data for the first specimen, the second indicators representing an aggregation of the pathology findings data for the first pathology slide and the second pathology slide;
while causing the display of the first display screen, receiving second user input specifying a change or addition to the diagnostic findings; and
in response to the second user input, updating the first screen display and the second screen display and the received or generated diagnostics finding data to reflect the change or addition.

11. The computer-implemented method of claim 10, further comprising:
while causing the display of the second display screen, receiving third user input specifying a change or addition to the diagnostic findings; and
in response to the third user input, updating the second screen display and the received or generated diagnostics finding data to reflect the change or addition.

12. The computer-implemented method of claim 10, further comprising:
receiving a further user input and in response to the further user input generating and/or displaying a report based on the updated received or generated diagnostics finding data.

13. A computer-implemented method, comprising:
receiving image data of images of a plurality of pathology slides, including a first image of a first of the pathology slides;
receiving pathology diagnostic findings data in relation to the plurality of pathology slides, including the first pathology slide, the pathology findings data indicating one or more parts of the first pathology slide associated with a pathology diagnostic finding defined by the pathology diagnostic findings data;
receiving first user input that specifies the first pathology slide;
in response to the first user input displaying a screen comprising the first image and graphical representation of the one or more parts of the first pathology slide associated with the pathology diagnostic finding.

14. The computer-implemented method of claim 13, further comprising receiving second user input and in response to the second user input displaying the first image without the graphical representation of the one or more parts of the first pathology slide associated with the pathology diagnostic finding.

15. The computer-implemented method of claim 14, further comprising receiving third user input and in response to the third user input displaying the first image with the graphical representation of the one or more parts of the first pathology slide associated with the pathology diagnostic finding.

16. The computer-implemented method of claim 13, wherein the graphical representation includes highlighted regions, corresponding to the one or more parts of the first pathology slide associated with the pathology diagnostic finding, overlayed on the first image.

17. The computer-implemented method of claim 13 further comprising:
receiving further user input indicating acceptance, rejection, or addition of a diagnostic finding;
in response to the further user input modifying the pathology diagnostic findings data to reflect the change indicated by the computer user.

18. The computer-implemented method of claim 13, further comprising:
receiving further user input specifying a said graphical representation and a magnify request;
in response to the further user input magnifying the region on the first image associated with the specified graphical representation.

19. The computer-implemented method of claim 10, further comprising:
displaying a summarised report of the diagnostic findings including a summarised report for each specimen, most important diagnostic findings across all specimens, and visual representation of areas affected by cancerous tissues.

20. The computer-implemented method of claim 19, wherein the visual representation of areas affected by cancerous tissues comprises a plurality of biopsy sample locations and diagnostic finding indicators associated with each biopsy sample location.

21. The computer-implemented method of claim 19, wherein the summarised report for each specimen includes a pattern bar chart where the pattern bar chart represents the overall area in the specimen affected by cancerous tissues and contains distinct areas for each type of cancerous pattern detected in the specimen.

22. (canceled)

23. (canceled)
